(19) United States
(12) Patent Application Publication
(10) Pub. No.: US 2026/0137731 A1
(43) Pub. Date: May 21, 2026

(54) APPLICATION OF BCG-BASED OMV TRAINED IMMUNITY INDUCER IN PREPARATION OF IMMUNOTHERAPEUTIC DRUG FOR SEPSIS

(71) Applicant: Children’s Hospital of Soochow University, Suzhou (CN)

(72) Inventors: Huiting ZHOU, Suzhou (CN); Haitao LV, Suzhou (CN); Sheng DING, Suzhou (CN)

(21) Appl. No.: 19/440,818

(22) Filed: Jan. 6, 2026

Related U.S. Application Data
(63) Continuation of application No. PCT/CN2025/135236, filed on Nov. 14, 2025.

(30) Foreign Application Priority Data
Nov. 18, 2024 (CN) 202411642653.X

(51) Int. Cl. A61K 35/74; A61K 9/50; A61P 31/00; C12N 1/20

(52) U.S. Cl. CPC A61K 35/74; A61K 9/5068; A61P 31/00; C12N 1/20

(57) ABSTRACT

An application of a Bacillus Calmette-Guérin (BCG)-based outer membrane vesicle (OMV) trained immunity inducer in preparation of an immunotherapeutic drug for sepsis is provided. The disclosure isolates and collects OMVs secreted by BCG bacteria to obtain a BCG-based OMV trained immunity inducer (designated as B-OMVs). The B-OMVs combines distinct advantages of both BCG and OMVs while circumventing their respective drawbacks. The developed B-OMVs exhibits characteristics such as being non-replicative, non-infectious, and of low toxicity, resulting in minimal impact on the organism and high safety. In addition, the B-OMVs demonstrates a more moderate yet effective immunomodulatory capacity. It can induce trained immunity, enhance the body's resistance to microbial infections, reduce the need for antibiotics, and more effectively improve the prognosis of sepsis.

### CROSS-REFERENCE TO RELATED APPLICATION

This application claims priority to Chinese Patent Application No. 202411642653.X, filed Nov. 18, 2024, which is herein incorporated by reference in its entirety.

### TECHNICAL FIELD

The disclosure relates to the technical field of biomedicines, and more particularly to an application of a Bacillus Calmette-Guérin (BCG)-based outer membrane vesicle (OMV) trained immunity inducer in preparation of an immunotherapeutic drug for sepsis.

### BACKGROUND

Sepsis is a clinical syndrome characterized by multiple organ dysfunction caused by a dysregulated host response to infection. The occurrence and pathogenesis of sepsis are accompanied by severe immune dysregulation. The disruption of body homeostasis resulting from an imbalance between pro-inflammatory and anti-inflammatory mechanisms is a primary cause of disease progression and even death in sepsis patients. Sepsis poses a serious threat to human life and health, yet effective treatment options are currently lacking. Beyond antimicrobial therapy targeting pathogenic microorganisms, most other treatments are non-specific symptomatic supportive treatment. Furthermore, the overuse of antibiotics has led to the emergence of multidrug-resistant bacteria, which is showing an increasing trend in clinical practice. The emergence of “superbugs” resistant to all existing antibiotics even presents a significant threat to global public health security. Therefore, developing a safe and effective novel drug for the treatment of sepsis is of paramount importance.

Trained immunity (TI) is an immunomodulatory strategy. Innate immune cells, represented by monocytes/macrophages, natural killer (NK) cells, and innate lymphoid cells (ILCs), interact with pathogen-associated molecular patterns (PAMPs) through pattern recognition receptors (PRRs), which triggers epigenetic and metabolomic reprogramming of the innate immune cells. Upon subsequent exposure to a non-specific stimulus, these cells mount a faster or more potent immune response to eliminate the stimulus and restore body homeostasis. This memory-like function of innate immunity is referred to as TI. TI possesses a powerful ability to resist pathogens such as viruses, bacteria, and fungi, enabling the body to rapidly restore homeostasis. Consequently, TI holds broad application prospects in the prevention and treatment of sepsis. Studies have shown that inducing TI in mice can enhance their pro-inflammatory response and bacterial clearance function following sepsis modeling, thereby rapidly eliminating pathogens and improving sepsis prognosis.

The attenuated bovine tuberculosis bacterium (Bacillus Calmette-Guérin, abbreviated as BCG) can induce TI and provide protective effects against infectious diseases and tumors. However, because BCG is a live attenuated vaccine with inherent immunogenicity, it can cause BCG disease, and in severe cases, even disseminated tuberculosis. These factors further limit the clinical application of BCG for inducing TI.

Bacterial outer membrane vesicles (OMVs) are nanoparticles naturally secreted by bacteria formed by outward budding of the bacterial membrane. They possess a structure and composition similar to the bacterial outer membrane and are enriched in PAMPs, enabling them to interact with immune cells for immunomodulation. However, OMVs derived from Gram-negative bacteria are rich in endotoxins, which can induce strong immunomodulatory responses. In some cases, they may even facilitate the bacterial infection process and trigger the onset of sepsis. In view of this, OMVs derived from Gram-negative bacteria must be attenuated to ensure safety before being applied in clinical therapy. Although various attenuation techniques, such as mineralization treatment and genetic engineering regulation, are currently employed, these methods still struggle to completely remove endotoxin components from OMVs.

### SUMMARY

In view of the limitations of the related art, an objective of the disclosure is to provide an application of a Bacillus Calmette-Guérin (BCG)-based outer membrane vesicle (OMV) trained immunity inducer in preparation of an immunotherapeutic drug for sepsis. The disclosure aims to combine the advantages of both BCG and OMVs, reduce their toxicity, and induce trained immunity more safely and effectively, thereby improving the prognosis of sepsis.

In order to achieve the above objective, the disclosure provides the following technical solutions.

In a first aspect, the disclosure provides a method for preparing a BCG-based OMV trained immunity inducer, including steps of:
S1, adding a BCG strain to a liquid culture medium for cultivation, collecting a bacterial culture, subsequently centrifuging the bacterial culture to collect a first supernatant, and filtering the first supernatant to remove bacteria and bacterial debris, thereby obtaining a filtrate;
S2, subjecting the filtrate to ultrafiltration to remove small molecular proteins, followed by concentration to obtain concentrated liquid; performing first centrifugation on the concentrated liquid to remove bacterial debris to thereby collect a second supernatant, and performing a second centrifugation on the second supernatant to obtain a sterile supernatant; and
S3, subjecting the sterile supernatant to ultracentrifugation to remove a third supernatant to obtain a resulting pellet, resuspending the resulting pellet in a phosphate-buffered saline (PBS) buffer to obtain a resulting suspension, and performing a washing centrifugation on the resulting suspension to obtain the BCG-based OMV trained immunity inducer.

In an embodiment, in the step S1, the liquid culture medium is M7H9 liquid medium.

In an embodiment, in the step S1, the cultivation is performed at a temperature of 35-37° C. with a rotational speed in a range of 200-300 revolutions per minutes (rpm).

In an embodiment, in the step S1, a value of an optical density at 600 nanometers (OD600) of the bacterial culture is in a range of 0.8-1.

In an embodiment, in the step S1, the centrifuging is performed at 3000-4000 g for 10-20 minutes.

In an embodiment, in the step S2, multiple of the concentration is in a range of 10-30 times.

In an embodiment, in the step S3, a rotational speed of the ultracentrifugation is in a range of 100,000-200,000 g and duration of the ultracentrifugation is in a range of 1-3 hours.

In a second aspect, the disclosure further provides an application of the BCG-based OMV trained immunity inducer prepared according to the above method in preparation of an immunotherapeutic drug for sepsis.

In an embodiment, the pharmaceutical formulation is selected from the group consisting of an injection, a tablet, a capsule, granules, pills, an oral liquid, and an emulsion.

In an embodiment, the immunotherapeutic drug further includes a pharmaceutically or pharmacologically acceptable carrier.

In an embodiment, the carrier includes one or more selected from the group consisting of a disintegrant, a diluent, a lubricant, a binder, a wetting agent, a flavoring agent, a suspending agent, a surfactant, and a preservative.

Compared with the related art, the disclosure provides the following beneficial effects.

The disclosure isolates and collects OMVs secreted by BCG bacteria to obtain the BCG-based OMV trained immunity inducer (designated as B-OMVs). The B-OMVs provided herein combines the distinct advantages of both BCG and OMVs while circumventing their respective drawbacks. The developed B-OMVs exhibits characteristics such as being non-replicative, non-infectious, and of low toxicity, resulting in minimal impact on the organism and high safety.

The B-OMVs provided by the disclosure demonstrates a more moderate yet effective immunomodulatory capacity. It can induce trained immunity, enhance the body's resistance to microbial infections, reduce the need for antibiotics, and more effectively improve the prognosis of sepsis.

### BRIEF DESCRIPTION OF DRAWINGS

FIG. 1 illustrates an electron micrograph of B-OMVs prepared in embodiment 1 of the disclosure.

FIGS. 2A-2C illustrate a set of comparative charts showing safety effects in different groups of mice. Specifically, FIG. 2A illustrates a comparison of survival rates among the different groups; FIG. 2B illustrates a comparison of organ injury-related markers among the different groups; and FIG. 2C illustrates a comparison of serum inflammatory cytokines among the different groups; where **P<0.01, ***P<0.001, ****P<0.0001.

FIGS. 3A-3D illustrates a set of comparative charts showing sepsis prognosis improvement effects in different groups of mice. Specifically, FIG. 3A illustrates a comparison of survival rates among the different groups; FIG. 3B illustrates a comparison of serum inflammatory cytokine levels among the different groups; FIG. 3C illustrates a comparison of organ injury-related markers among the different groups; and FIG. 3D illustrates a comparison of bacterial loads in organs among the different groups; where *P<0.05, **P<0.01, ***P<0.001.

### DETAILED DESCRIPTION OF EMBODIMENTS

The disclosure is further described in detail below through specific illustrated embodiments, but the disclosure is not limited to the following embodiments.

It should be noted that, unless otherwise specified, chemical reagents involved in the disclosure are purchased from commercial sources.

The BCG strain used in the disclosure was donated by the Suzhou Center for Disease Control and Prevention, China, and the Escherichia coli strain used was ATCC 8099.

### Embodiment 1

A method for preparing a BCG-based OMV trained immunity inducer includes the following steps.
S1. A BCG strain is added to M7H9 liquid culture medium and cultured on a shaking incubator at 37° C. and 250 rpm. When the OD600 value of the bacterial culture reaches 0.8, the bacterial culture is collected and centrifuged at 3,450 g for 15 minutes. The supernatant (i.e., first supernatant) is collected and filtered through a 0.45 micrometers (μm) filter to completely remove bacteria and large debris, thereby obtaining a filtrate.
S2. The filtrate is ultrafiltered using a 100 kilodaltons (kDa) molecular weight cutoff (MWCO) filter to remove small molecular proteins. The ultrafiltered solution is concentrated 20-fold to obtain concentrated liquid. A first centrifugation is performed on the concentrated liquid at 4,000 g for 15 minutes to remove large debris, and the supernatant (i.e., second supernatant) is collected. A second centrifugation is performed on the supernatant at 15,000 g for 30 minutes to collect the supernatant as a sterile supernatant.
S3. The sterile supernatant is subjected to ultracentrifugation at 150,000 g for 2 hours in an ultracentrifuge to remove the supernatant (i.e., third supernatant), so as to obtain a resulting pellet. The resulting pellet is resuspended in 500 microliters (μL) of PBS buffer to obtain a resulting suspension. A washing centrifugation is performed on the resulting suspension at 20,000 g for 15 minutes to obtain the BCG-based OMV trained immunity inducer (designated as B-OMVs).

### Embodiment 2

A method for preparing a BCG-based OMV trained immunity inducer includes the following steps.
S1. A BCG strain is added to M7H9 liquid culture medium and cultured on a shaking incubator at 37° C. and 250 rpm. When the OD600 value of the bacterial culture reaches 0.9, the bacterial culture is collected and centrifuged at 3,450 g for 15 minutes. The supernatant (i.e., first supernatant) is collected and filtered through a 0.45 μm filter to completely remove bacteria and large debris, thereby obtaining a filtrate.
S2. The filtrate is ultrafiltered using a 100 kDa MWCO filter to remove small molecular proteins. The ultrafiltered solution is concentrated 20-fold to obtain concentrated liquid. A first centrifugation is performed on the concentrated liquid at 4,000 g for 15 minutes to remove large debris, and the supernatant (i.e., second supernatant) is collected. A second centrifugation is performed on the supernatant at 15,000 g for 30 minutes to collect the supernatant as a sterile supernatant.
S3. The sterile supernatant is subjected to ultracentrifugation at 150,000 g for 2 hours in an ultracentrifuge to remove the supernatant (i.e., third supernatant), so as to obtain a resulting pellet. The resulting pellet is resuspended in 500 μL of PBS buffer to obtain a resulting suspension. A washing centrifugation is performed on the resulting suspension at 20,000 g for 15 minutes to obtain the BCG-based OMV trained immunity inducer.

### Embodiment 3

A method for preparing a BCG-based OMV trained immunity inducer includes the following steps.
S1. A BCG strain is added to M7H9 liquid culture medium and cultured on a shaking incubator at 37° C. and 250 rpm. When the OD600 value of the bacterial culture reaches 1.0, the bacterial culture is collected and centrifuged at 3,450 g for 15 minutes. The supernatant (i.e., first supernatant) is collected and filtered through a 0.45 μm filter to completely remove bacteria and large debris, thereby obtaining a filtrate.
S2. The filtrate is ultrafiltered using a 100 kDa MWCO filter to remove small molecular proteins. The ultrafiltered solution is concentrated 20-fold to obtain concentrated liquid. A first centrifugation is performed on the concentrated liquid at 4,000 g for 15 minutes to remove large debris, and the supernatant (i.e., second supernatant) is collected. A second centrifugation is performed on the supernatant at 15,000 g for 30 minutes to collect the supernatant as a sterile supernatant.
S3. The sterile supernatant is subjected to ultracentrifugation at 150,000 g for 2 hours in an ultracentrifuge to remove the supernatant (i.e., third supernatant), so as to obtain a resulting pellet. The resulting pellet is resuspended in 500 μL of PBS buffer to obtain a resulting suspension. A washing centrifugation is performed on the resulting suspension at 20,000 g for 15 minutes to obtain the BCG-based OMV trained immunity inducer.

### Comparative Embodiment 1

A method for preparing an E. coli-based OMV biological preparation includes the following steps.
S1. An E. coli strain is added to M7H9 liquid culture medium and cultured on a shaking incubator at 37° C. and 250 rpm. When the OD600 value of the bacterial culture reaches 0.8, the bacterial culture is collected and centrifuged at 3,450 g for 15 minutes. The supernatant is collected and filtered through a 0.45 μm filter to completely remove bacteria and large debris, thereby obtaining a filtrate.
S2. The filtrate is ultrafiltered using a 100 kDa MWCO filter to remove small molecular proteins. The ultrafiltered solution is concentrated 20-fold to obtain concentrated liquid. A first centrifugation is performed on the concentrated liquid at 4,000 g for 15 minutes to remove large debris, and the supernatant is collected. A second centrifugation is performed on the supernatant at 15,000 g for 30 minutes to collect the supernatant as a sterile supernatant.
S3. The sterile supernatant is subjected to ultracentrifugation at 150,000 g for 2 hours in an ultracentrifuge to remove the supernatant, so as to obtain a resulting pellet. The resulting pellet is resuspended in 500 μL of PBS buffer to obtain a resulting suspension. A washing centrifugation is performed on the resulting suspension at 20,000 g for 15 minutes to obtain the E. coli-based OMV biological preparation (designated as E-OMVs).

Compared with the embodiment 1, the comparative embodiment 1 uses the E. coli strain instead of the BCG strain.

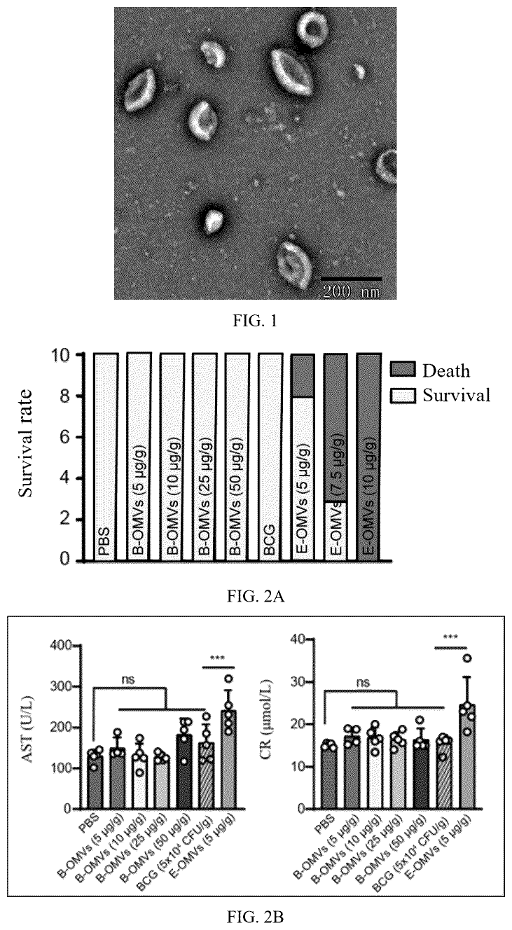

The electron micrograph of the B-OMVs prepared in the embodiment 1 of the disclosure is shown in FIG. 1. As can be seen from FIG. 1, the B-OMVs prepared in the embodiment 1 of the disclosure have a nanostructure with a particle size of less than 200 nm.

Tests are conducted on the B-OMVs prepared in the embodiment 1 of the disclosure, as detailed below.

### Test Example 1 Evaluation of B-OMVs Safety

The safety of B-OMVs is evaluated in C57 BL/6 mice by intraperitoneal injection of different doses of B-OMVs and E-OMVs (the biological preparation from the comparative embodiment 1), with assessment based on survival rate, organ injury-related markers, and serum inflammatory cytokines. The specific experimental steps are as follows.

B-OMVs are administered at doses of 5 μg/g, 10 μg/g, 25 μg/g, and 50 μg/g; E-OMVs are administered at doses of 5 μg/g, 7.5 μg/g, 10 μg/g, and 15 μg/g; BCG is administered at a dose of 5×10⁴ colony-forming units (CFU) per mouse. Mice are divided into groups according to the above dosages, with five mice per group. Each group is injected intraperitoneally with the corresponding dosage. After injection, the mortality of mice is observed every 24 hours, and the experiment is terminated after 7 days. The number of dead mice in each group is recorded and statistically analyzed.

B-OMVs are administered at doses of 5 μg/g, 10 μg/g, 25 μg/g, and 50 μg/g; E-OMVs are administered at doses of 5 μg/g; BCG (Bacillus Calmette-Guérin) is administered at a dose of 5×10⁴ CFU per mouse. Mice are divided into groups accordingly, with five mice per group. Each group is injected intraperitoneally with the corresponding dosage. Serum samples are collected 24 hours post-injection. Interleukin-6(IL-6 ) and tumor necrosis factor-alpha (TNF-α) levels are detected by enzyme-linked immunosorbent assay (ELISA), and aspartate aminotransferase (AST) and creatinine (CR) levels are measured using a clinical chemistry analyzer.

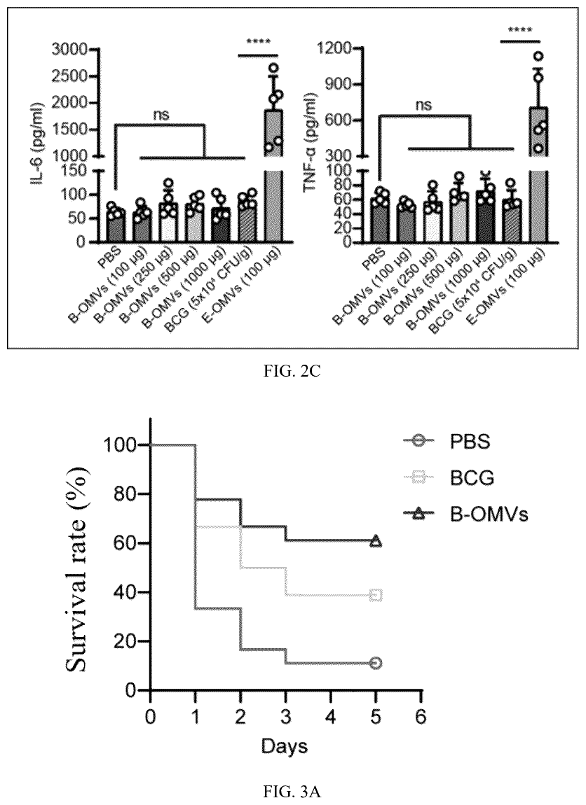

The results are shown in FIGS. 2A-2C. As can be seen from FIG. 2 A, the safety of B-OMVs is significantly higher than that of E-OMVs. High-dose (50 μg/g) administration of B-OMVs does not cause mouse death. As can be seen from FIG. 2B and FIG. 2C, inflammatory cytokines and organ injury-related markers in the B-OMVs and BCG groups are essentially normal. In contrast, low-dose (5 μg/g) administration of E-OMVs induces severe inflammatory reactions, leading to partial mouse death, elevated inflammatory cytokines, and increased organ injury markers. Thus, the B-OMVs provided by the disclosure exhibit high safety characteristics and show promising potential for clinical application.

### Test Example 2 Evaluation of B-OMVs-Induced Trained Immunity in Improving Sepsis Prognosis in Mice

C57 BL/6 mice are intraperitoneally injected with B-OMVs or BCG to induce trained immunity, with PBS injection serving as a control. Subsequently, a cecal ligation and puncture (CLP) is performed to establish a mouse sepsis model. The protective effect of pre-injection with B-OMVs against sepsis in mice is evaluated by comparing survival rates, inflammatory cytokine levels, organ injury-related markers, and bacterial burden.

Specific experimental steps are as follows.

### Grouping and Treatment of C57BL/6 Mice

Fifty-four C57BL/6 mice aged 6-8 weeks are randomly divided into three groups: PBS-CLP, BCG-CLP, and B-OMVs-CLP, with 18 mice per group. The PBS-CLP group is intraperitoneally injected with 0.5 mL PBS as a control. The BCG-CLP group is intraperitoneally injected with 5×10⁴ CFU per mouse. The B-OMVs-CLP group is intraperitoneally injected with 5 μg/g. After drug administration, the mice rest for 3 days.

### Establishment of Mouse Sepsis Model and Treatment

To induce the mouse sepsis model, CLP surgery is performed on all mice from the above groups. The procedure is conducted as follows. Mice are anesthetized, the abdominal skin is shaved, and the surgical site is disinfected with iodine. A small incision (approximately 1 cm) is made in the lower abdomen. The cecum is gently exteriorized using forceps. A 50% ligature is surgically tied around the cecum, followed by a puncture with a needle. An appropriate amount of intestinal contents is extruded through the puncture site. The cecum is then returned to the abdominal cavity, and the incision is sutured. Subcutaneous injection of 1 mL sterile normal saline pre-warmed to 37° C. is administered for fluid resuscitation.

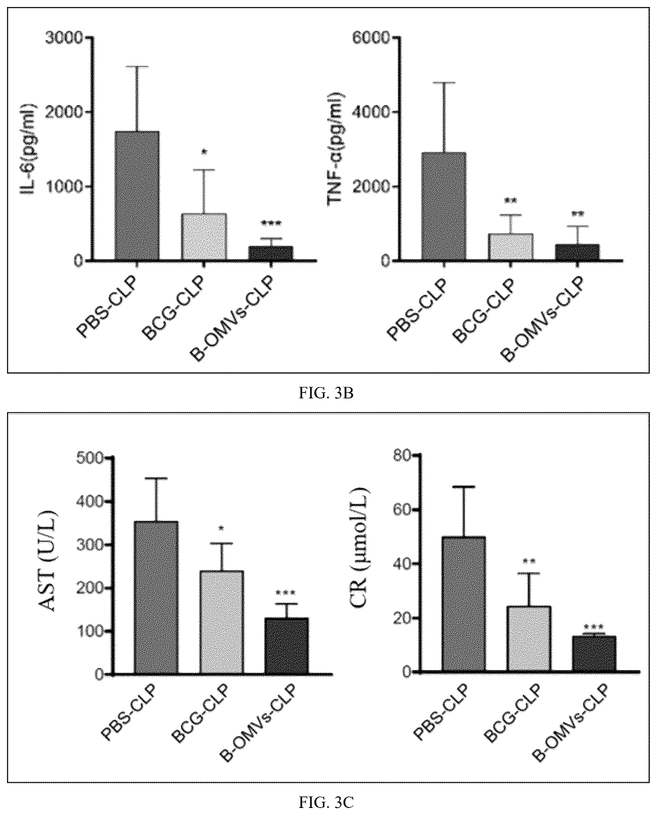

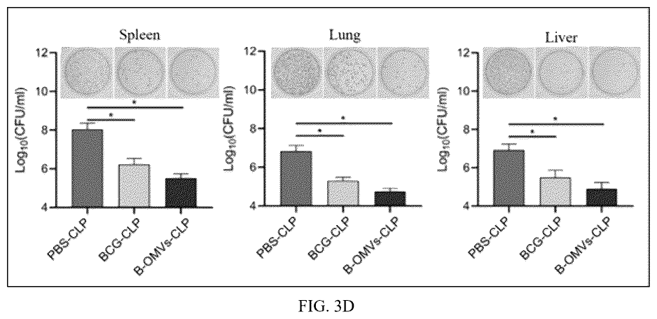

Twenty-four hours after CLP modeling, the survival status of C57BL/6 mice in each group is recorded every 24 hours. Survival curves are analyzed statistically using the Log-Rank test. The results are shown in FIG. 3A.

Twenty-four hours after CLP modeling, whole blood is collected from mice and serum is isolated. Levels of IL-6 and TNF-α in the serum are detected by ELISA. The results are shown in FIG. 3B. AST and CR levels are measured using a clinical chemistry analyzer. The results are shown in FIG. 3C.

Organs including liver, spleen, and lungs are collected from mice 24 hours after CLP modeling. Each organ is homogenized under sterile conditions. The homogenate is resuspended in an equal volume of PBS. The suspension is plated on Luria-Bertani (LB) agar plates. Bacterial growth is observed after 24 hours of incubation. The results are shown in FIG. 3D.

As can be seen from FIGS. 3A-3D, compared with the BCG group, the B-OMVs group exhibits significantly higher survival rates, significantly lower levels of inflammatory cytokines, markedly reduced organ injury, and significantly decreased bacterial burden. It is demonstrated that the B-OMVs provided by the disclosure, through inducing trained immunity, enhance early anti-inflammatory and antibacterial functions in mice, thereby enabling rapid elimination of pathogens at an early stage, preventing infection dissemination, and improving the prognosis of septic mice.

Finally, it should be noted that the above embodiments do not limit the disclosure in any form. For those skilled in the art, various modifications and improvements can be made on the basis of the disclosure. Therefore, any modifications or improvements made without departing from the spirit of the disclosure shall fall within the scope of protection claimed by the disclosure.

What is claimed is:

1. An application of a Bacillus Calmette-Guérin (BCG)-based outer membrane vesicle (OMV) trained immunity inducer in preparation of an immunotherapeutic drug for sepsis, wherein the BCG-based OMV trained immunity inducer is prepared by a method comprising steps of:
S1, adding a BCG strain to a liquid culture medium for cultivation, collecting a bacterial culture, subsequently centrifuging the bacterial culture to collect a first supernatant, and filtering the first supernatant to remove bacteria and bacterial debris, thereby obtaining a filtrate;
S2, subjecting the filtrate to ultrafiltration to remove small molecular proteins, followed by concentration to obtain concentrated liquid; performing first centrifugation on the concentrated liquid to remove bacterial debris to thereby collect a second supernatant, and performing a second centrifugation on the second supernatant to obtain a sterile supernatant; and
S3, subjecting the sterile supernatant to ultracentrifugation to remove a third supernatant to obtain a resulting pellet, resuspending the resulting pellet in a phosphate-buffered saline (PBS) buffer to obtain a resulting suspension, and performing a washing centrifugation on the resulting suspension to obtain the BCG-based OMV trained immunity inducer.

2. The application as claimed in claim 1, wherein in the step S1, a value of an optical density at 600 nanometers (OD₆₀₀) of the bacterial culture is in a range of 0.8-1.

3. The application as claimed in claim 1, wherein in the step S2, multiple of the concentration is in a range of 10-30 times.

4. The application as claimed in claim 1, wherein in the step S3, a rotational speed of the ultracentrifugation is in a range of 100,000-200,000 g and duration of the ultracentrifugation is in a range of 1-3 hours.

5. The application as claimed in claim 1, wherein a pharmaceutical formulation of the immunotherapeutic drug is selected from the group consisting of an injection, a tablet, a capsule, granules, pills, an oral liquid, and an emulsion.

6. The application as claimed in claim 1, wherein the immunotherapeutic drug further comprises a pharmaceutically or pharmacologically acceptable carrier.

7. The application as claimed in claim 6, wherein the carrier comprises one or more selected from the group consisting of a disintegrant, a diluent, a lubricant, a binder, a wetting agent, a flavoring agent, a suspending agent, a surfactant, and a preservative.
